(19) United States
(12) Patent Application Publication
(10) Pub. No.: US 2025/0073145 A1
(43) Pub. Date: Mar. 6, 2025

(54) DEODORANT COMPOSITIONS

(71) Applicant: The Procter & Gamble Company, Cincinnati, OH (US)

(72) Inventors: Edward Dewey Smith, III, Mason, OH (US); David Frederick Swaile, Cincinnati, OH (US); Steven Edward Witt, Morrow, OH (US); Min Wei, Mason, OH (US); Shane Whitaker, Liberty Township, OH (US)

(21) Appl. No.: 18/825,441

(22) Filed: Sep. 5, 2024

Related U.S. Application Data
(60) Provisional application No. 63/580,448, filed on Sep. 5, 2023.

(51) Int. Cl. A61K 8/36; A61Q 15/00

(52) U.S. Cl. CPC A61K 8/361; A61Q 15/00; A61K 2800/30; A61K 2800/591; A61K 2800/75

(57) ABSTRACT

A stick deodorant composition that includes a solvent with at least one polyhydric alcohol and a structurant that includes a blend of fatty acids. The fatty acid blend has a lower amount of sodium palmitate (C16:0), as compared to current deodorant products with stearic acid.

### FIELD OF THE INVENTION

The present disclosure relates to deodorant compositions and methods relating thereto. More particularly, an aluminum-free deodorant stick with a fatty acid structurant.

### BACKGROUND

Many consumers are seeking more natural, aluminum-free deodorant offerings that are often mostly free of silicones. These products are formulated to combat body odor by inhibiting the growth of odor-causing bacteria and providing aesthetically pleasing fragrance throughout the day. One way to reduce microorganism growth is to formulate deodorant products so when they are applied to the skin, the skin surface has a pH outside the pH range of optimal microorganism growth. These products can also be transparent or translucent to provide protection on the skin while being invisible so they can be more aesthetically pleasing to wear.

Some consumers prefer aluminum-free glycol-based deodorant sticks over other forms because they are convenient, quick-drying, transparent or translucent, and are known for being effective at limiting odor. However, irritation caused by deodorant products is a concern for some individuals, especially for those who shave their underarms regularly because the application of deodorant immediately after shaving can exacerbate irritation. Fragrances, high pH, and certain solvents are known culprits in deodorant-related irritation experienced by some users.

Thus, there is a continuing challenge to formulate an aluminum-free, fragrance deodorant stick with low irritation while maintaining sufficient pH control throughout the day to inhibit bacterial growth. Furthermore, the deodorant stick needs to have thermal stability, so it does not melt during shipping, handling, or use, and can be processed using standard commercial methods for manufacturing stick deodorant.

### SUMMARY

A stick deodorant composition comprising: (a) a solvent compartment comprising a solvent comprising at least one polyhydric alcohol; (b) a blend of fatty acids comprising C16, C18, C20, and C22 carbon chain lengths; wherein the fatty acid blend comprises≤4% C16 fatty acid, as a percentage of the total fatty acids; wherein the deodorant composition is substantially free of aluminum.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows a chart representing a model that was constructed from the data representing the blend of fatty acids and the processing temperature.

### DETAILED DESCRIPTION

Many companies (see select examples in Table 1, below), manufacture and sell aluminum-free deodorant compositions in a glycol-based stick, often referred to as a gel stick. Some consumers prefer the gel stick product form because in addition to being aluminum-free, it is long-lasting, dries quickly, is moisturizing, is translucent or transparent, and can be effective at reducing odor throughout the day. The gel stick deodorant is predominantly solvent and also contains a metal salt of a fatty acid as the structurant.

The solvent, which can comprise about 80-90% of the total formulation, frequently includes dipropylene glycol, propylene glycol, glycerin, and/or water. The solvents can be miscible with each other and are also generally miscible with the fragrance, forming what can be considered a separate phase of the deodorant stick (i.e., solvent phase or solvent compartment).

The fatty acid and/or metal salt of the fatty acid is added to stick deodorant products as a structurant and can exist as a distinct phase that forms a hard gel. The metal salt of the fatty acid can affect the hardness and the melt point of the composition. It can also help reduce underarm sweating and provide antimicrobial benefits. However, fatty acids by themselves can irritate some users' skin. Additionally, fatty acids can also enhance skin penetration of active agents, which can aid in drug delivery, if desired, and can increase penetration of unwanted substances, particularly fragrances. Therefore, if deodorant sticks contain fatty acid structurants, it may be desirable for structuring agents comprising fatty acids to be insoluble, both from the standpoint of enhanced ability to provide structure, and to minimize their ability to act as skin penetration enhancers.

Aluminum-free deodorant sticks are most effective if they can change the pH under the arm throughout the day. A pH between 5 and 8 provides optimal conditions for bacterial growth and subsequently underarm malodor. Therefore, it is beneficial for a deodorant to keep the pH of the underarm above at least 8.

Procter & Gamble and several other companies currently market aluminum-free deodorant products that are predominantly solvent and contain a sodium stearate structurant. Table 1, below, shows the ingredient list for eight commercial deodorant stick products purchased in Cincinnati, OH, USA area in March of 2022, that include sodium stearate as the only or primary structurant. Portions of seven deodorant sticks from Table 1 were assayed according to the Deodorant Stick Fatty Acid Method, described hereafter. The results from this test are shown in Table 2, below. In Table 2, the FA % for C16:0, C18:0, C22:0, C20:0 and total fatty acids is % weight of the deodorant product and the C16 proportion is the percent of the total fatty acids that is sodium palmitate (C16:0), as determined by the Deodorant Stick Fatty Acid Method, described hereafter. The total fatty acids are calculated by adding the total fatty acids that were measured by the Deodorant Stick Fatty Acid Method. The pH is determined by the pH Test Method, described hereafter. Table 2 also includes titratable base in micromoles acid per gram solvent, which can be determined by the Deodorant Titratable Base Method, described hereafter.

Table 2 shows that even though all seven commercial products list sodium stearate as the structuring agent, the sodium stearate contains a substantial level of other fatty acids. For example, the compositions contain a substantial amount of palmitate (C16:0, ranging from 1.066-1.834% of the entire composition and 28% to 30% of the fatty acids), behenate (C22:0, ranging from 0.318-0.835% of the entire composition), and stearate (C:18:0).

Furthermore, all the products in Table 2 have a basic pH, between 9.1 and 10.6. Gel sticks can have a pH in this range because sodium stearate requires a pH of greater than about 9 to fully ionization the fatty acid, in particular the longer chain length portions, and it is also beneficial to formulate with a basic pH to inhibit microbial growth.

TABLE 1
Examples of Commercially Available Aluminum-
Free Stick Deodorant Products

| Product | Ingredient List |
|---|---|
| Secret ® Clear | Dipropylene Glycol, Water, Propylene Glycol, |
| Waterlily | Sodium Stearate, Poloxamine 1307, PPG-3 |
| Aluminum Free | Myristyl Ether, Fragrance, Tetrasodium EDTA |
| Deodorant | |
| Dove ® Beauty | Dipropylene Glycol, Water, Glycerin, Propylene |
| 0% Aluminum | Glycol, Sodium Stearate, Poloxamine 1307, |
| Coconut & Pink | Fragrance, Butyrospermum Parkii (Shea) Butter, |
| Jasmine | Aloe Barbadensis Leaf Juice Powder, Chamomilla |
| Deodorant Stick | Recutita (Matricaria) Flower Extract, Stearic |
| | Acid, Disodium EDTA, Simethicone, BHT |
| Axe Apollo ® | Dipropylene Glycol, Simethicone, Water, |
| Deodorant Stick | Propylene Glycol, Sodium Stearate, C12 15 Alkyl |
| | Benzoate, Fragrance, Disodium EDTA, BHT, Green 5, |
| | Yellow 10 |
| Axe ® Phoenix | Dipropylene Glycol, Simethicone, Water, Propylene |
| Deodorant Stick | Glycol, Sodium Stearate, C12-15 Alkyl Benzoate, |
| | Fragrance (Parfum), Disodium EDTA, BHT, Ext |
| | Violet 2, Blue 1 |
| Dove | Dipropylene Glycol, Water, Glycerin, Propylene |
| Men + Care ® | Glycol, Sodium Stearate, Poloxamine 1307, Fragrance, |
| Clean Comfort | Stearic Acid, Disodium EDTA, BHT, Simethicone, |
| Deodorant Stick | Alpha-Isomethyl Ionone, Citral, Citronellol, Coumarin, |
| | Hexyl Cinnamal, Limonene, Linalool |
| Dove | Dipropylene Glycol, Water, Glycerin, Propylene |
| Men + Care ® | Glycol, Sodium Stearate, Poloxamine 1307, Fragrance, |
| Extra Fresh | Stearic Acid, Disodium EDTA, BHT, Simethicone, |
| Deodorant Stick | Citronellol, Hexyl Cinnamal, Limonene, Linalool. |
| Old Spice ® | Dipropylene Glycol, Water, Propylene Glycol, Sodium |
| Men's Deodorant | Stearate, Fragrance, Poloxamine 1307, Ppg-3 Myristyl |
| Aluminum-Free | Ether, Tetrasodium EDTA, Green 6. |
| Wilderness with | |
| Lavender | |
| Every Man | Propanediol, Water, Sodium Stearate, Polyglyceryl-10 |
| Jack ® | Caprylate/Caprate, Caprylyl Glycol, Fragrance, |
| Cedarwood | Sodium Chloride, Cetyl Alcohol, Aloe Barbadensis |
| Men's Deodorant- | Leaf Extract, Hamamelis Virginiana (Witch Hazel) |
| Aluminum Free | Leaf Extract, Gossypium Herbaceum (Cotton) Seed |
| | Extract, Calendula Officinalis Flower Extract, |
| | Commiphora Myrrha Resin Extract, Usnea Barbata |
| | (Lichen) Extract, Citrus Grandis (Grapefruit) Seed |
| | Extract, Chamomilla Recutita (Matricaria) Flower |
| | Extract, Rosmarinus Officinalis (Rosemary) Oil, Salvia |
| | Officinalis (Sage) Oil, Juniperus Virginiana |
| | (Cedarwood) Oil |

TABLE 2
Fatty Acids in Stick Deodorant Composition

| | | | | | | | Total | C16 |
|---|---|---|---|---|---|---|---|---|
| | | Titrateable | C16:0 | C18:0 | C22:0 | C20:0 | Fatty | Proportion |
| Product | pH | Base | FA % | FA % | FA % | FA % | Acids | (%) |
| Secret ® Clear | 10.5 | | 1.356 | 1.863 | 0.613 | 1.09 | 4.92 | 28 |
| Waterlily | | | | | | | | |
| Aluminum Free | | | | | | | | |
| Deodorant | | | | | | | | |
| Dove ® Beauty 0% | 9.1 | | 1.701 | 2.252 | 0.674 | 0.984 | 5.61 | 30 |
| Aluminum Coconut | | | | | | | | |
| & Pink Jasmine | | | | | | | | |
| Deodorant Stick | | | | | | | | |
| Axe Apollo ® | 10.5 | | 1.084 | 1.521 | 0.318 | 0.920 | 3.84 | 28 |
| Deodorant Stick | | | | | | | | |
| Axe ® Phoenix | 10.5 | | 1.066 | 1.350 | 0.374 | 1.011 | 3.80 | 28 |
| Deodorant Stick | | | | | | | | |
| Dove Men + Care ® | 10.5 | | 1.834 | 2.244 | 0.835 | 1.129 | 6.04 | 30 |
| Clean Comfort | | | | | | | | |
| Deodorant Stick | | | | | | | | |
| Dove Men + Care ® | 10.2 | | 1.802 | 2.217 | 0.835 | 1.155 | 6.00 | 30 |
| Extra Fresh | | | | | | | | |
| Deodorant Stick | | | | | | | | |
| Old Spice ® Men's | 10.6 | 51.2 | 1.236 | 1.627 | 0.580 | 0.986 | 4.42 | 28 |
| Deodorant | | | | | | | | |
| Aluminum-Free | | | | | | | | |
| Wilderness with | | | | | | | | |
| Lavender | | | | | | | | |
| Every Man Jack ® | | 76.4 | | | | | | |
| Cedarwood Men's | | | | | | | | |
| Deodorant - | | | | | | | | |
| Aluminum Free | | | | | | | | |

Deodorant sticks, like those in Table 1, are generally made by adding the sodium stearate to the solvents, heating it until the sodium stearate melts, and mixing until it is uniform. The molten mixture is then poured into packaging and cooled to precipitate the gel network, which contains the sodium stearate, ultimately making a solid gel stick.

It was believed that to provide structure, the structuring agent is expected to be essentially insoluble. To test this theory, a portion of the deodorant sticks from Table 1 were centrifuged to separate the solvent compartment from the insoluble components and the solvent compartment was analyzed for individual fatty acid components using the Deodorant Solvent Phase Fatty Acid Method, described hereafter.

In Table 3, the FA % for C16:0, C18:0, C22:0, C20:0 and total fatty acids are expressed as a weight percentage of the total solvent. The C16 proportion was calculated as the C16 proportion of the total fatty acids measured in the solvent and expresses as a percentage. The C16 proportion in the solvent compartment was between 66%-79%, whereas the C16 only comprises between 28% and 30% of the added total fatty acid structurant component in Table 2. This shows that C16 has a relatively high solubility as compared to other fatty acids in the deodorant sticks. The soluble C16 in Table 3 is calculated by estimating that each stick product contains 90% total solvent, and it was calculated that approximately 22.9% to 43.1% of the C16 component intended for structuring, is actually soluble in the solvent compartment of the stick.

TABLE 3
% Soluble FA in Solvent Phase

| | | | | | | | Soluble |
|---|---|---|---|---|---|---|---|
| | | | | | Total | C16 | C16 (% |
| | C16:0 | C18:0 | C22:0 | C20:0 | Fatty | Proportion | of all |
| Product | FA % | FA % | FA % | FA % | Acids | (%) | C16) |
| Secret ® Clear | 0.4180 | 0.1720 | 0.0090 | 0.0360 | 0.635 | 66 | 27.7 |
| Waterlily Aluminum | | | | | | | |
| Free Deodorant | | | | | | | |
| Dove ® Beauty 0% | 0.5220 | 0.1660 | 0.0070 | 0.0230 | 0.718 | 73 | 27.6 |
| Aluminum Coconut | | | | | | | |
| & Pink Jasmine | | | | | | | |
| Deodorant Stick | | | | | | | |
| Axe Apollo ® | 0.3580 | 0.1330 | 0.0000 | 0.0230 | 0.514 | 70 | 29.7 |
| Deodorant Stick | | | | | | | |
| Axe ® Phoenix | 0.3390 | 0.1190 | 0.0030 | 0.0260 | 0.487 | 70 | 28.6 |
| Deodorant Stick | | | | | | | |
| Dove Men + Care ® | 0.5590 | 0.1570 | 0.0060 | 0.0210 | 0.743 | 75 | 27.4 |
| Clean Comfort | | | | | | | |
| Deodorant Stick | | | | | | | |
| Dove Men + Care ® | 0.4590 | 0.1060 | 0.0040 | 0.0130 | 0.582 | 79 | 22.9 |
| Extra Fresh | | | | | | | |
| Deodorant Stick | | | | | | | |
| Old Spice ® Men's | 0.4797 | 0.2073 | 0.0179 | 0.034 | 0.705 | 68 | 43.1 |
| Deodorant | | | | | | | |
| Aluminum-Free | | | | | | | |
| Wilderness with | | | | | | | |
| Lavender | | | | | | | |
| Every Man Jack ® | 0.7376 | 0.402 | 0.000 | 0.000 | 1.1396 | 65 | — |
| Cedarwood Men's | | | | | | | |
| Deodorant - | | | | | | | |
| Aluminum Free | | | | | | | |

It was surprising to find fatty acids, that were previously thought to be insoluble, in the solvent phase of every stick deodorant tested. This means that not only do the deodorant compositions have a high pH, but they have a higher amount of soluble base than previously expected, the fatty acids being dissolved in their ionized form being bases.

High pH deodorants can elevate skin surface pH for a period of up to 6 hours or longer and elevated skin pH can also lead to impairment of the stratum corneum barrier increasing the potential for irritation. The process of returning the skin surface pH to its natural acidity involves primarily generation of new acids in and on the stratum corneum, such as lactic acid from sweat, and pyrrolidone carboxylic acid and uroconic acid, which are generated in the skin. The amount of acid the skin must produce to return skin to its baseline pH is therefore a function of the pH of the applied product, how much product is applied, and the amount of soluble base (i.e., the product's buffering capacity). Therefore, if a deodorant product has a high buffering capacity, it will require more acid from the skin to return to baseline than a product with a lower buffering capacity. Therefore, there is a need to provide a deodorant product that has a high pH, to obtain the benefits thereof, while at the same time a low buffering capacity, to minimize the potential for skin barrier related irritation.

The data summarized in Table 2 and Table 3 shows that there is a significant amount of soluble fatty acid in the deodorant stick. Since the soluble fatty acids are soluble and deprotonated, reducing soluble fatty acids in the deodorant stick could provide two benefits: reduced skin penetration enhancement effects and enhanced skin pH recovery.

Next, a cumulative irritation patch test (CIPT) was run to determine if stick deodorant products made with sodium behenate (C22:0) were less irritating than stick deodorant products made from sodium stearate, which had a range of chain lengths. Deodorant product is applied to a patch which is adhesively attached to the skin on the back or arm, which can then be partially or fully occluded, for a period of five days. Dermatological assessments are performed daily including irritation scoring. Patches are reapplied after evaluations, but testing is discontinued at any site when irritation reaches a prescribed level. The frequency of discontinued sites for a treatment in a study is therefore also a measurement of irritation (TOXICOL. SCI., 148:101-107 (2015), doi: 10.1093/toxsci/kfv158).

Table 4 shows the formulas for Example A-C and skin patch study results for each example. Example A includes 5.5% sodium stearate (OP-200V from Hallstar® Beauty Products Co., USA), which is known to have the following distribution of carbon chain lengths: 2% sodium myristate (C14), 27% sodium palmitate (C16), 35% sodium stearate (C18), and 36% of sodium arachidate (C20)+sodium behenate (C22). The ultracentrifuged solvent of Ex. A performed essentially the same as Ex. A, which demonstrates that the soluble components are responsible for the observed effects.

As shown in Table 4, the composition of Ex. B is similar to Ex. A, except it contains sodium behenate structurant instead of sodium stearate structurant. Ex. A has over 40 times the amount of soluble C16 fatty acid (i.e., 0.463% in Ex. A and 0.011% in Ex. B) and over 10 times the amount of soluble fatty acid (i.e., 0.69% in Ex. A and 0.059% in Ex. B). Ex. B was less irritating as compared to Ex. A by all measurements. For instance, Ex. A had 66% dropped sites and Ex. B had 34%, which indicates that Ex. B was less irritating as compared to Ex. A and may be preferred by some consumers.

TABLE 4
Deodorant Stick Examples and Skin Patch Study Results

| | Ex. A | Ex. B | Ultracentrifuged |
|---|---|---|---|
| | (wt. %) | (wt. %) | Solvent of Ex. A |
| Sodium stearate¹ | 5.5 | 0 | |
| Sodium behenate | 0 | 5.0 | |
| Dipropylene glycol | 53 | 53 | |
| Propylene glycol | 20 | 20 | |
| Glycerin | 0 | 0 | |
| Water | 18 | 18 | |
| Fragrance | 3 | 3 | |
| Miscellaneous | 0.5 | 1 | |
| % soluble C16 FA | 0.463 | 0.011 | 0.463 |
| % total soluble FA | 0.69 | 0.059 | 0.694 |
| Skin Patch Study Results | | | |
| % dropped sites | 66 | 34 | 64 |
| Total Mean Irritation score | 5.65 | 3.94 | 5.65 |
| % sites scored as zero | 8 | 16 | 7 |
| irritation | | | |

¹OP-200V (Hallstar ® Beauty Products Co., USA)

Based only on the data in Table 3, one may want to eliminate fatty acids with 16 or 18 carbons because they make up almost all the soluble fatty acids. However, if the fatty acid structurant contains too many C20, C22, and C24 fatty acids, the processing temperature can be too high to allow for standard commercial deodorant processing methods and the product could be too opaque to be consumer preferred.

Table 8, below, shows seven examples (described in Table 5 and Table 7, below) and a commercial product (described in Table 6, below). Some consumers complain when the melt point is below 55° C. because the deodorant stick can start to melt and deform if it gets too hot, for instance, if a consumer leaves their gym bag in their car on a warm summer day. However, in order to manufacture the deodorant sticks, the peak melt temperature can be below 80° C. to allow standard commercial deodorant processing methods. Peak melt temperature is determined according to the Peak Melt Temperature Test Method as follows: using a Differential Scanning Calorimeter to melt the deodorant stick in a standard container heated at 5° C./minute to melt the sample. The peak melt temperature is the temperature at the heat flow minimum (a negative value) when plotted as exothermic heat flow vs temperature.

Table 8, below, shows that the examples with a ratio of sodium palmitate (C16) to sodium stearate (C18)+sodium behenate (C22) of greater than 0 and less than 1:2, alternatively less than 1:4, alternatively less than 1:10, and alternatively less than 1:20 can provide a consumer preferred melt point while still having a peak melting temperature that allows standard commercial processing.

TABLE 5
Chassis

| Material | (wt. %) |
|---|---|
| Propylene Glycol | 19.0 |
| Dipropylene glycol | 44.7 |
| Water | QS |
| Stearic Acid | 0-4%, see Table 7 |
| Behenic Acid | 0-5%, see Table 7 |
| 10% sodium hydroxide | amount required to provide 1:1 mole ratio with |
| | total stearic acid and behenic acid blend |
| Poloxamine 1307 | 3.0 |
| Tetrasodium EDTA | 0.5 |
| PPG-3 myristyl ether | 1.4 |
| Fragrance | 3.0 |

TABLE 6

| Product | Ingredient List |
|---|---|
| Old Spice ® Red Collection | Dipropylene Glycol, Water, Propylene |
| Swagger Scent Deodorant for | Glycol, Sodium Stearate, Fragrance, |
| Men (hereinafter | PPG-3 Myristyl Ether, Tetrasodium |
| “Swagger”) | EDTA, Blue 1 |

TABLE 7
Example

| | |
|---|---|
| 1 | Chassis (Table 5) + 4% Stearic Acid + 1% Behenic Acid |
| 2 | Chassis (Table 5) + 3% Stearic Acid + 2% Behenic Acid |
| 3 | Chassis (Table 5) + 2.5% Stearic Acid + 2.5% Behenic Acid |
| 4 | Chassis (Table 5) + 2% Stearic Acid + 3% Behenic Acid |
| 5 | Chassis (Table 5) + 1% Stearic Acid + 4% Behenic Acid |
| 6 | Chassis (Table 5) + 5% Behenic Acid |
| 7 | Chassis (Table 5) + 5% Sodium Behenate |

TABLE 8

| | | | | | | | DSC | | DSC | DSC | |
|---|---|---|---|---|---|---|---|---|---|---|---|
| | | | | | | Ratio of | Onset of | DSC Peak | Onset of | Peak | |
| | C16 | C18 | C20 | C22 | C24 | C16 to | Crystallization | Crystallization | Melt | Melt | Hardness |
| Ex. | % | % | % | % | % | C18 + C22 | (° C.) | (° C.) | (° C.) | (° C.) | (Pens) |
| 1 | 4.08 | 76.59 | 1.36 | 17.60 | 0.36 | 1:23 | 42.83 | 41.11 | 59.76 | 60.92 | 93.0 |
| 2 | 3.17 | 58.14 | 2.73 | 35.24 | 0.72 | 2:59 | 49.02 | 47.08 | 63.94 | 64.35 | 89.4 |
| 3 | 2.71 | 48.90 | 3.41 | 44.08 | 0.90 | 3:103 | 50.35 | 48.68 | 65.32 | 66.33 | 89.0 |
| 4 | 2.25 | 39.64 | 4.09 | 52.93 | 1.08 | 1:41 | 55.78 | 54.6 | 71.75 | 72.41 | 97.9 |
| 5 | 1.33 | 21.10 | 5.47 | 70.66 | 1.45 | 1:69 | 61.26 | 60.52 | 74.83 | 76.07 | 104.8 |
| 6 | 0.40 | 2.52 | 6.84 | 88.43 | 1.81 | 1:226 | 67.99 | 67.35 | 80.96 | 81.47 | 113.3 |
| 7 | 0.00 | 0.72 | 12.96 | 86.32 | 0 | 0 | 68.35 | 67.35 | 77.27 | 77.88 | 108.8 |
| Swagger | 27.34 | 36.80 | 21.63 | 14.23 | 0 | 15:28 | 38.69 | 36.86 | 56.65 | 56.98 | 93.4 |

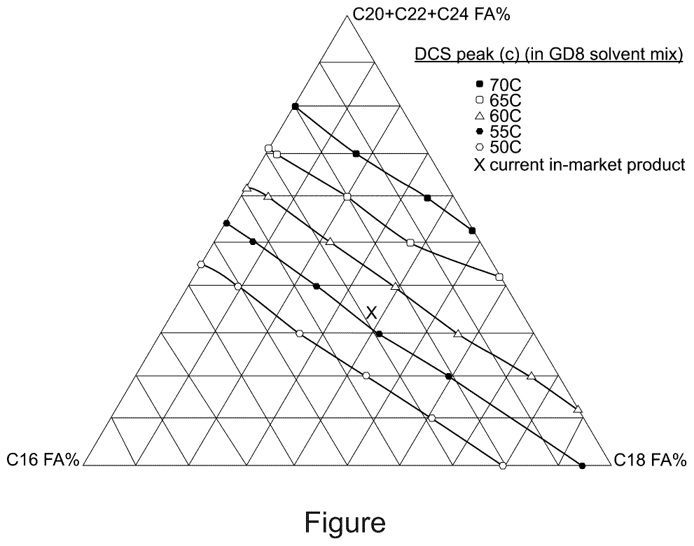

FIG. 1 shows a chart representing a model that was constructed from the data in Table 8. The model shows that if the blend included 20% C16, as a percentage of the total fatty acid salts, the blend could also include 60% C20+C22+C24 and 20% C18 with a processing temperature of about 65° C. If the blend included 10% C16, as a percentage of the total fatty acid salts, the blend could also include 50% C20+C22+C24 and 40% C18 with a processing temperature of about 66° C.

It also may be advantageous to use a mixture of fatty acids with carbon chain lengths from C16 to C22 to get a consumer preferred translucency. Stick deodorant users may want a more translucent product, which can signal that the product will appear invisible or nearly invisible on the user's skin. Table 9, below, shows the % T for five examples and one commercial product, all fatty acids having different carbon chain length distributions from C16 to C22, as measured by the Transmittance Test Method, described herein. Table 9, shows that compositions with more sodium behenate, tend to be more opaque. Thus, it could be advantageous to use some C16 to achieve a translucency that some consumers prefer. Transparency can be measured by the Transmittance Test Method described herein.

Table 9, below, also shows the titratable base in micromoles acid per gram solvent as measured by the Deodorant Titratable Base Method, described herein. The Method measures total base, without discriminating between the type of base, which can include deprotonated, soluble fatty acids and amine salts (e.g., EDTA). The soluble fatty acids can be the primary titratable base because they are abundant, and both soluble and deprotonated, while the insoluble fatty acids can be relatively inert. Thus, reducing soluble fatty acids in the deodorant stick could provide two benefits: reduced skin penetration enhancement effects and enhanced skin pH recovery thereby reducing irritation.

TABLE 9

| | Old | | | | | |
|---|---|---|---|---|---|---|
| | Spice ® | | | | | |
| | Men's | | | | | |
| | Deodorant | | | | | |
| | Aluminum | Ex. 1 | Ex. 2 | Ex. 4 | Ex. 5 | Ex. 6 or 7 |
| | (Table 1) | (Table 7) | (Table 7) | (Table 7) | (Table 7) | (Table 7) |
| Titratable | 51.2 | 39.8 | 37.3 | 38.2 | 37.0 | 28.0 |
| Base | | | | | | |
| % T | 16.3 | 5.0 | 0.4 | 0.2 | 0 | 0 |
| (600 nm) | | | | | | |

### Deodorant Stick Composition

The deodorant compositions of the present invention may have a hardness measured by a penetration value of at most about 120 units, as determined by the test method detailed below. In some embodiments, the hardness may be from about 80 units to about 120 units, alternatively from about 85 units to about 115 units as measured by the Hardness Test Method, described hereafter. The deodorant stick composition can have a pH from about 9 to about 10.7, alternatively from about 9.5 to about 10, according to the pH Test Method.

The stick deodorant formulation can have a melt point greater than 55° C., alternatively greater than 57° C., alternatively greater than 60° C., alternatively greater than 65°. The stick deodorant composition can be translucent with a % T at 600 nm of greater than 0, alternatively ≥0.2, alternatively ≥0.4, alternatively ≥1, alternatively ≥ to 5, alternatively greater than ≥10, alternatively ≥12, alternatively ≥15, as determined by the Transmittance (% T) Test Method, described hereafter.

### Solvents

The composition can be predominantly solvent. In one example, the solvent can be from about 50% to about 90%, alternatively from about 60% to about 90%, alternatively from about 70% to about 90%, alternatively from about 75% to about 90%, alternatively from about 80% to about 90%, and alternatively from about 85% to about 90%. The solvents can be miscible. The solvents can be chosen from polyhydric alcohol, water, or mixtures thereof.

The composition can be predominantly solvent. In one example, the solvent can be from about 50% to about 90%, alternatively from about 60% to about 90%, alternatively from about 70% to about 90%, alternatively from about 75% to about 90%, alternatively from about 80% to about 90%, and alternatively from about 85% to about 90%. The solvents can be miscible. The solvents can include one or more polyhydric alcohols and water. The solvents can be polar.

The solvent can include polyhydric alcohol. Polyhydric alcohols may include, but are not limited to propylene glycol, dipropylene glycol, tripropylene glycol, low molecular weight polypropylene glycols, 1,3 propanediol, ethylene glycol, diethylene glycol, triethylene glycol, butylene glycol, hexylene glycol, trimethylene glycol, 1,2 pentanediol, 1,2 hexanediol, butantediol, glycerol, sorbitol, and polyethylene glycol including PEG-4 and PEG-8, or mixtures thereof. The composition can include more than 30% polyhydric alcohol, alternatively more than 40%, alternatively more than 50%, alternatively more than 60%, and alternatively more than 70%.

The solvent can include water. The composition can have from about 1% to about 30% water, alternatively from about 3% to about 25%, and alternatively from about 5% to about 20%.

The solvents can include one or more polyhydric alcohols and water. The weight ratio of water to polyhydric alcohol can be from about 1:10 to about 1:1, alternatively from about 1:8 to about 4:5, alternatively from about 1:7 to about 7:8, alternatively from about 1:6 to about 3:4, alternatively from about 1:5 to about 2:3, alternatively from about 1:5 to about 1:2, and alternatively from about 1:5 to about 1:3.

The deodorant stick can use less than 50 micromoles acid per gram solvent titratable base, alternatively less than 45, and alternatively less than 40, as determined by the Deodorant Titratable Base Method, described herein.

### Fatty Acid Structurants

The fatty acid structurant can form a gel network. Many commercial products include a sodium stearate structurant. When deodorant sticks gelled with sodium stearate are applied to the skin, the gel network is believed to serve as an organic reservoir, remaining on the skin surface, which reduces the ability of irritants such as fragrance molecules to penetrate the stratum corneum, ameliorating the irritation potential of the fragrances.

Commercial grade sodium stearate is chemically heterogeneous. It is common for its fatty acid mixtures to comprise a mixture of sodium palmitate, sodium stearate, and sodium behenate and still retain the nomenclature “sodium stearate.” In some current products, sodium palmitate is a preferred component of commercial sodium stearate for its ability to provide clarity to the sticks. A commercial example of sodium stearate is designated OP 200 manufactured by Hallstar, Inc. (California, USA) which comprises a mixture of about 2% sodium myristate (C14), 27% sodium palmitate (C16), 35% sodium stearate (C18), and 36% of sodium arachidate (C20)+sodium behenate (C22) and is labeled sodium stearate on commercial deodorant packages.

As described herein, it can be desirable to use a mixture of carbon chain lengths of the fatty acid structurants from C16 to C22 to optimize processing temperature, melting point, hardness, and % T. The mixture may contain sodium palmitate (C16) and contain≤30% C16 as a percentage of the total fatty acid, alternatively≤28%, alternatively≤25%, alternatively≤20%, alternatively ≤15% as a percentage of the total fatty acid salts, alternatively≤12%, alternatively≤10%, alternatively≤9%, alternatively≤8%, alternatively≤7%, alternatively≤6%, alternatively≤5%, alternatively≤4%, alternatively≤3%, or alternatively≤2%, as determined by the Deodorant Stick Fatty Acid Method, described hereafter.

The mixture can contain C18 fatty acid and contain ≥30% C18 as a percentage of the total fatty acid salts, alternatively ≥35%, alternatively ≥40%, alternatively ≥45%, alternatively ≥50%, alternatively >55%, as determined by the Deodorant Stick Fatty Acid Method, described hereafter.

The mixture can contain C20 fatty acid and contain ≥20% C20 as a percentage of the total fatty acid salts, alternatively ≥25%, alternatively ≥27%, alternatively ≥30%, alternatively ≥35%, alternatively >40%, as determined by the Deodorant Stick Fatty Acid Method, described hereafter.

The mixture can contain C22 fatty acid and contain ≥10% C22 as a percentage of the total fatty acid salts, alternatively ≥12%, alternatively ≥15%, alternatively ≥18%, alternatively ≥20%, as determined by the Deodorant Stick Fatty Acid Method, described hereafter.

The solvent compartment can contain≤0.5% C16 fatty acid by weight of the solvent compartment, alternatively≤0.4%, alternatively≤0.3%, alternatively≤0.25%, alternatively≤0.25%, alternatively≤0.15%, alternatively≤0.1%, alternatively≤0.05%, as determined by the Deodorant Solvent Phase Fatty Acid Method, described hereafter.

The mixture can contain a ratio of sodium palmitate/(sodium stearate+sodium behenate) of ≤1:3, alternatively≤1:4, alternatively≤1:5, alternatively≤1:6, alternatively≤1:7, alternatively ≤1:8, alternatively≤1:9, alternatively≤1:10, and alternatively≤1:12, as determined by the Deodorant Stick Fatty Acid Method, described hereafter.

### Fragrance

Fragrance is a common component of deodorants, and fragrance molecules have varying degrees of polarity, being somewhat or partially soluble in more than one phase. The extent to which any fragrance molecule or mixture is soluble in different locations (phases) within a deodorant stick is called its partitioning behavior and is governed by thermodynamic principles governing solvent-solute interactions. Fragrance molecules can be at least partly soluble in more than one component of a deodorant stick formulation.

The stick deodorant composition can include any fragrance that is cosmetically acceptable. The fragrance may be a liquid at room temperature. A wide variety of chemicals are known as fragrances, including aldehydes, ketones, and esters. More commonly, naturally occurring plant and animal oils and exudates comprising complex mixtures of various chemical components are known for use as fragrances. Generally, the fragrance can be present at a level from about 0.1% to about 30%, from about 0.25% to about 20%, from about 0.5% to about 10%, or from about 1% to about 5%, by weight of the composition.

### Additional Antimicrobials

The deodorant stick may include one or more antimicrobial compositions. For example, antimicrobials may include, without being limited to, piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, undecylenoyl phenylalanine (commercially available as Sepiwhite™), Propylene Glycol (and) Ethylhexylglycerin (and) Octenidine HCl (commercially available as Sensidin™), baking soda, partially carbonated magnesium hydroxide, magnesium carbonate hydroxide, cinnamon essential oil, cinnamon bark essential oil, cinnamic aldehyde, and combinations thereof.

### Chelator

The deodorant compositions may comprise a chelator. Specific and/or additional chelators in the present invention may include, but are not limited to, diethylenetriaminepentaacetic acid (DTPA), diethylenetriaminepentakis (methylenephosphonic acid) (DTPMP), desferrioxamine, their salts and combinations thereof, EDTA, DPTA, EDDS, enterobactin, desferrioxamine, HBED, and combinations thereof. The amount of chelator, by weight of composition, may be from about 0.05% to about 4%.

### Solubilizer

The composition can contain a solubilizer. A suitable solubilizer can be, for example, a surfactant, such as a no-foaming or low-foaming surfactant. Suitable surfactants are nonionic surfactants, cationic surfactants, amphoteric surfactants, zwitterionic surfactants, and mixtures thereof.

Suitable solubilizers include, for example, hydrogenated castor oil, polyoxyethylene 2 stearyl ether, polyoxyethylene 20 stearyl ether, and combinations thereof. One suitable hydrogenated castor oil that may be used in the present composition is polyoxyethylene hydrogenated castor oil.

When the solubilizing agent is present, it is typically present at a level of from about 0.01% to about 5%, alternatively from about 0.01% to about 3%, alternatively from about 0.05% to about 1%, alternatively from about 0.01% to about 0.05%, by weight of the composition.

### Preservatives

The composition can include a preservative. The preservative is included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis (5-(p-chlorophenyl) biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis (hydroxymethyl) 2,5-dioxo-4-imidazolidinyl]-N,N′-bis (hydroxy-methyl) urea, commonly known as diazolidinyl urea, available under the trade name Germall® II from Sutton Laboratories, Inc.; N,N″-methylenebis {N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formal-dehyde; glutaraldehyde; polyaminopropyl biguanide, available under the trade name Cosmocil CO® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative can range from about 0.0001% to about 0.5%, alternatively from about 0.0002% to about 0.2%, alternatively from about 0.0003% to about 0.1%, by weight of the composition.

### Methods of Use

The deodorant compositions described herein may be topically applied to the axilla or other area of the skin in any known or otherwise effective method for controlling malodor associated with perspiration. These methods comprise applying to the axilla or other area of the human skin an effective amount of the deodorant composition, typically about 0.1 gram per axilla to about 2.0 gram per axilla. The stick deodorant composition is generally a leave-on composition that can provide lasting odor protection and freshness. The composition can be substantially free of or free of aluminum salts, parabens, dyes, and/or talc. The deodorant stick can be applied without white marks, in other words, it can go on clear and stay clear.

### Definitions

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

Except as otherwise noted, the articles “a”, “an”, and “the” mean “one or more”.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “majority” refers to greater than about 51% of the stated component or parameter.

“Substantially free of” refers to about 2% or less, about 1% or less, or about 0.1% or less of a stated ingredient. “Free of” refers to no detectable amount of the stated ingredient or thing.

“Gel network” refers to the structurant component of the deodorant which hardens it into a stick form.

### Test Methods

### Deodorant Stick Fatty Acid Method

A 7% (w/v) solution of the deodorant stick in chloroform is made using an analytical balance to determine an accurate weight of the sample. To this 625 μg of tritridecanoin internal standard is added via pipette using a stock solution also in chloroform. A small aliquot (100-250 ul) of this solution is then transferred to an autosamplers vials with an equal amount of derivatizing reagent. The derivatizing reagent is N,O-bistrifluoroacetamide with 1% trimethylchlorosilane (BSTFA+1% TCMS). Once mixed this solution is capped and allow to derivatize at room temperature for 12 hours. A similar reference standard is made with the relevant fatty acids (e.g. palmitic acid, stearic acid, arachidonic acid and behenic acid).

The above solutions are then injected on a gas chromatograph capable of a 1 ul liquid split injection. A 0.25 mm ID capillary column with 0.25 um polydimethyl siloxane station phase is used with a hydrogen mobile phase for the separation. The injector and detector are maintained at 350C with the oven ramping from 80C to 350C. Flame ionization is used to detect each peak. Using the relative response from each analyte to the internal standard a response factor is calculated for each analyte in the reference standard and from this the amounts for each sample normalizing to the sample weight. Some of these samples contain polypropyleneglycol-3 (PPG-3) which interferes with the palmitic acid peak. This is corrected for by using one of the other peaks from the PPG-3 that does not coclute. The PPG-3 is run by itself to determine the relative ratio of the interfering peak with the reference peak. Then this is applied to each sample to determine the contribution of the PPG-3 to the palmitic acid peak response. This is then subtracted, and a corrected palmitic acid is determined. Results are reported as percent by weight.

### Deodorant Solvent Phase Fatty Acid Method

Using Beckman Optima Ultracentrifuge with SW32Ti swing bucket rotor and appropriate tubes, filled without air by deacrating in a conventional centrifuge, centrifuge deodorant stick product at 50,000 rpm, 35° C. for 12 h. The denser solvent compartment will transparently separate from solids due to its density. Cut the tube open and decant the solvent compartment obtained into a separate container, avoiding sampling contact with other layers, to obtain about 500-1,000 mg of solvent compartment. Prepare a 7% w/v solution of the solvent compartment in chloroform using an analytical balance. Add 625 μg of tritridecanoin internal standard via pipette using a stock solution also in chloroform. Transfer a small aliquot (100-250 ul) of this solution to an autosampler vial with an equal amount of derivatizing reagent. The derivatizing reagent is N,O-bistrifluoroacetamide with 1% trimethylchlorosilane (BSTFA+1% TCMS). Once mixed this solution is capped and allow to derivatize at room temperature for 12 hours. Prepare a similar reference standard made with each relevant fatty acids (e.g. palmitic acid, stearic acid, arachidonic acid and behenic acid).

Inject the solutions on a gas chromatograph capable of a 1 μl liquid split injection. A 0.25 mm ID capillary column with 0.25 μm polydimethyl siloxane station phase is used with a hydrogen mobile phase for the separation. The injector and detector are maintained at 350° C. with the oven ramping from 80° C. to 350° C. Flame ionization is used to detect each peak. Using the relative response from each analyte to the internal standard a response factor is calculated for each analyte in the reference standard and from this the amounts for each sample normalizing to the sample weight. If a sample contains an interfering chemical such as polypropyleneglycol-3-myristoyl ether which can interfere with the palmitic acid peak, this is corrected for by using one of the other peaks from the PPG-3 ether that does not coclute. The PPG-3 ether is run by itself to determine the relative ratio of the interfering peak with the reference peak. Then this is applied to each sample to determine the contribution of the PPG-3 ether to the fatty acid peak response. This is then subtracted to correct the result. Results are reported as percent by weight.

### Deodorant Titratable Base Method

The Deodorant Titratable Base Method measures the amount of acid required to titrate the solvent compartment of a basic deodorant, to the pH of the skin, which is about 5. All pH measurements are obtained at ambient temperature (21-23° C.) using a calibrated pH meter. A digital 3-place balance is used to weigh all materials for accuracy. Prepare a dilute hydrochloric acid titrant using 6N hydrochloric acid in deionized water, to between 10-15 micromoles HCl per g titrant, to use for the titration. Use the equation 6*weight 6N hydrochloric acid solution added in grams/1100 g solution per liter/total weight of water+acid solution added in grams*1,000,000 micromoles per mole to obtain the titrant concentration in micromoles acid per gram titrant solution. Using Beckman Optima Ultracentrifuge with SW32Ti swing bucket rotor and appropriate tubes, filled without air by deaerating in a conventional centrifuge, centrifuge deodorant stick product at 50,000 rpm, 35° C. for 12 h. The denser solvent compartment will transparently separate from solids due to its density. Cut the tube open and decant the solvent obtained into a separate container, avoiding sampling contact with other layers, to obtain at least about 1 gram of solvent compartment. Solvent compartment from more than one tube can be combined to obtain the mass needed. Prepare at least 2 grams of 1:1 dilution of the solvent compartment obtained with deionized water in a 20 ml scintillation vial having a diameter only slightly larger than a pH probe. Record the masses of solvent compartment and water in the vial.

Dry the pH probe completely using a tissue, insert it into the vial to immerse the probe, and measure and record the pH after the reading stabilizes for at least 30 seconds in the second decimal point. This is the solvent compartment pH. Carefully remove the probe in a manner to retain essentially all the liquid in the vial, without spilling. Do not dry the probe, do not touch the probe tip to any surfaces, and do not allow liquid drops to drip from the probe surface, so that all of the mass of liquid in the vial is available for the next measurement. Add about 0.25-0.75 grams of the dilute acid titrant to the vial, recording the added weight. Carefully swirl the mixture for 5-10 seconds, reinsert the probe, and use the probe to continue to stir the mixture until homogeneous, for about 10-15 seconds. Allow the pH reading to stabilize as before and record the pH. Continue to sequentially add more acid titrant stepwise following the same procedure each time, recording the added titrant amount, until at least 2 measurements are obtained below pH 5 to complete the measurement, typically requiring about 10 or more dilution steps. The solution in the vial may become turbid during the titration due to for example fragrances that become insoluble, which is normal. A graph of the measured pH vs total micromoles acid added per gram of solvent phase should be a smooth titration curve. The Titratcable Base of the deodorant is the total amount of acid added in micromoles that is required to reduce the pH of 1 gram of solvent compartment to pH 5 and is obtained by interpolation of the two titration points proximal to the pH 5 measurement, one point above and one below pH 5, expressed as micromoles acid per gram solvent phase.

### Hardness Test Method-Penetration Measurement for Deodorant Finished Products

The penetration test is a physical test method that provides a measure of the firmness of waxy solids and extremely thick creams and pastes with penetration values not greater than 250 when using a needle for D1321. The method is based on the American Society for Testing and Materials Methods D-5, D1321 and D217 and DIN 51 579 and is suitable for all solid antiperspirant and deodorant products.

A needle or polished cone of precisely specified dimensions and weight is mounted on the bottom of a vertical rod in the test apparatus. The sample is prepared as specified in the method and positioned under the rod. The apparatus is adjusted so that the point of the needle or cone is just touching the top surface of the sample. Consistent positioning of the rod is critical to the measured penetration value. The rod is then released and allowed to travel downward, driven only by the weight of the needle (or cone) and the rod. Penetration is the tenths of a millimeter travelled following release.

### Apparatus Suggested Type (or Equivalent)

Penetrometer with Timer Penetrometer Suitable For ASTM D-5 and D-1321 methods; Examples: Precision or Humboldt Universal Penetrometer (Humboldt Manufacturing, Schiller Park, IL USA) or Penetrometer Model PNR 10 or PNR12 (Petrolab USA or PetroTest GmbH).

Penetration Needles Antiperspirant or deodorant solids can use:
Needles as specified for ASTM Method D-5, NIST Certified, Fisher Scientific #01-512.
Needles as specified for ASTM Method D 1321/DIN 51 579, Officially certified, Taper-Tipped needle, No. H-1310, Humboldt Mfg.

General Instructions—All Penetrometers—Keep the instrument and needles/probes clean at all times, free from dust and grime. When not in use, store needles in a suitable container to avoid damage. Periodic calibration should confirm:

Electronic Timer is correctly set. Verify against an independent stopwatch if unsure.

Shaft falls without visible signs of frictional resistance.

Ensure the total weight of the shaft and needle is 50±0.2 grams when the shaft is in free

fall. Note: for modern, automated or digital systems this may be performed automatically and confirmed through annual calibration.

At time of use confirm:

Electronic Timer is correctly set to 5.0 seconds.

The appropriate needle is installed and is clean, straight and without obvious defects (visual inspection)

The penetrometer is level and the shaft is clean, straight and falls freely (visual inspection)

Once level, avoid shifting the position of the unit to maintain level.

### Sample Preparation and Measurement

1. On a deodorant stick that has cooled ambiently to a temperature between 22° C. and 26° C. for at least 24 hours, slice off top ½ inch of product to achieve a flat surface with a wire cutter drawn across the upper lip of the canister. This will be the reading for the Top of the stick.
2. For the first sample to be tested, lubricate the ASTM D-5 needle by gently wiping with a lint-free tissue coated with a small amount of the product to be tested. This small amount is typically taken from the shaved top.
3. Place the canister in the appropriate location for the measurement. Locate the sample so the needle will penetrate the product 9-11 mm from the inside of the canister wall on the long axis.
4. Using the coarse and fine adjustments, align the height of the penetrometer mechanism head so that the point of the penetrating needle is just touching the surface of the sample. A weak light at the side of the penetrometer which casts a shadow of the needle on the surface of the sample may be helpful in determining this contact. When a light area on the sample cannot be seen at the end of the tip of the needle's shadow, the needle height over the sample is correctly adjusted. The light should not be strong enough to heat or melt the sample surface. The needle should be just close enough to scratch the sample surface.
5. Perform the penetration measurement at this location by releasing the needle. Record the result.
6. Repeat Steps 2 through 4 at the other test point, i.e., at the other point 9-11 mm inside of the canister wall on the long axis.

To report results, units for penetration are tenths of a millimeter ( 1/10 mm=100 microns). Report the average results of at least 4 total measurements from 2 different sticks. Report the average result of the measurements to the nearest tenth of a millimeter.

### pH Test Method

### Materials and Apparatus

PH Meter (calibrated)
Balance (3 place) for measuring finished product & DI water
Water bath with ice (to lower samples to 26° C.±1.0° C.) range for measurement
Cutter or razor for slicing finished product in a thin film to maximize surface area
Wide-mouth jar with lid for samples, 40-50 ml range
36° C. convection oven
Deionized (DI) water

### Sample Preparation

First, slice deodorant stick across top of package to expose fresh product for sampling. Then, very thinly slice (<1 mm, 0.40 g±0.1 g) a shaving of finished product into jar. Calculate water necessary for 1% solution in DI water and add. Close the lid. Do not mix, stir, or shake beyond what is necessary for handling & movement of vessel around lab.

Next, place solution (DI water and sample) in the jar in 36° C. convection oven for 1 hour. Remove from oven.

Before pH measurement, use ice water bath to adjust temperature of solution between 26° C.±1.0° C. Remove lid and use probe to check temp as needed.

### pH Measurement

Place the electrode in the 1% solution, gently swirling solution by hand while waiting for reading to stabilize. Record pH once stable (ensuring temperature requirements are met).

### Transmittance (% T) Test Method

% T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. The deodorant stick is a 26 mm width stick, outside a container if present.

### EXAMPLES

The data and examples provided herein help illustrate the stick deodorant compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The deodorant stick compositions described herein were made by heating all ingredients to above their melt point and using typical mixing procedures known to those of ordinary skill in the art. All ingredients, excluding fragrance, are heated to 85° C. When all materials are homogeneous the formulation is held at the elevated temperature for a minimum of 10 minutes. The temperature is reduced to then reduced, at which point the fragrance is added. The formulation is poured into a canister suitable for a gel or stick underarm product and then allowed to cool.

### Combinations:

A. A stick deodorant composition comprising:
   a. a solvent compartment comprising a solvent comprising at least one polyhydric alcohol;
   b. a blend of fatty acids comprising C16, C18, C20, and C22 carbon chain lengths;
   wherein the fatty acid blend comprises ≤30% C16 fatty acid, as a percentage of the total fatty acids, preferably ≤28%, more preferably ≤25%, more preferably ≤20%, more preferably ≤15%, more preferably ≤12%, more preferably ≤10%, more preferably ≤9%, more preferably <8%, more preferably ≤7%, more preferably ≤6%, more preferably ≤5%, more preferably ≤4%, more preferably ≤3%, or more preferably ≤2%, as determined by the Deodorant Stick Fatty Acid Method described herein;
   wherein the deodorant composition is substantially free of aluminum.
B. The stick deodorant composition according to paragraph A, wherein a ratio of the C18 to the C20+C22 is from 1:1 to 6:1, preferably from 3:1 to 5:1, as determined by the Deodorant Stick Fatty Acid Method described herein.
C. The stick deodorant composition according to paragraphs A-B, wherein a ratio of the C16 to the C18+C22 fatty acids is ≤1:3, preferably ≤1:4, more preferably ≤1:5, more preferably ≤1:6, more preferably ≤1:7, more preferably ≤1:8, more preferably ≤1:9, more preferably ≤1:10, and more preferably ≤1:12, as determined by the Deodorant Stick Fatty Acid Method described herein.
D. The stick deodorant composition according to paragraphs A-C, wherein the fatty acid comprises ≥30% of the C18, as a percentage of the total fatty acid, preferably ≥35%, more preferably ≥40%, more preferably ≥45%, more preferably ≥50%, more preferably >55%, as determined by the Deodorant Stick Fatty Acid Method described herein.
E. The stick deodorant composition according to paragraphs A-D, wherein the fatty acid comprises ≥20% of the C20, as a percentage of the total fatty acid, preferably ≥25%, more preferably ≥27%, more preferably ≥30%, more preferably ≥35%, more preferably >40%, as determined by the Deodorant Stick Fatty Acid Method described herein.
F. The stick deodorant composition according to paragraphs A-E, wherein the fatty acid comprises ≥10% of the C22, as a percentage of the total fatty acid salts, preferably >12%, more preferably ≥15%, more preferably ≥18%, more preferably ≥20%, as determined by the Deodorant Stick Fatty Acid Method described herein.
G. The stick deodorant composition according to paragraphs A-F, wherein the solvent compartment comprises≤0.5% of the C16 fatty acid, by weight of the solvent compartment, preferably ≤0.4%, more preferably ≤0.3%, more preferably ≤0.25%, more preferably ≤0.25%, more preferably ≤0.15%, more preferably ≤0.1%, more preferably ≤0.05%, as determined by the Deodorant Solvent Phase Fatty Acid Method described herein.
H. The stick deodorant composition according to paragraphs A-G, wherein the composition comprises a pH from 9 to 10.5, preferably from 9.5 to 10, as determined by the pH Test Method described herein.
I. The stick deodorant composition according to paragraphs A-H, wherein the composition comprises a hardness from 80 units to 120 units, preferably from 85 units to 115 units, as determined by the Hardness Test Method described herein.
J. The stick deodorant composition according to paragraphs A-I, wherein the composition comprises from 50% to 90% of the solvent, preferably from 60% to 90%, more preferably from 75% to 90%, and even more preferably from 80% to 90%.
K. The stick deodorant composition according to paragraphs A-J, wherein the solvent is polar.
L. The stick deodorant composition according to paragraphs A-K, wherein the solvent comprises more than 30% polyhydric alcohol, preferably more than 40%, more preferably more than 50%, more preferably more than 60%, and more preferably more than 70%.
M. The stick deodorant composition according to paragraphs A-L, wherein the solvent further comprises from 1% to 30% water, more preferably from 3% to 25%, and more preferably from 5% to 20%.
N. The stick deodorant composition according to paragraphs A-M, wherein the weight ratio of water to polyhydric alcohol can be from 1:10 to 1:1, preferably from 1:8 to 4:5, more preferably from 1:7 to 7:8, more preferably from 1:6 to 3:4, more preferably from 1:5 to 2:3, more preferably from 1:5 to 1:2, and more preferably from 1:5 to 1:3.
O. The stick deodorant composition according to paragraph N, wherein the polyhydric alcohol and the water are miscible.
P. The stick deodorant composition according to paragraphs A-O, wherein the polyhydric alcohol is chosen from propylene glycol, dipropylene glycol, tripropylene glycol, low molecular weight polypropylene glycols, 1,3 propanediol, ethylene glycol, diethylene glycol, triethylene glycol, butylene glycol, hexylene glycol, trimethylene glycol, 1,2 pentanediol, 1,2 hexanediol, butantediol, glycerol, sorbitol, and polyethylene glycol including PEG-4 and PEG-8, or mixtures thereof.
Q. The stick deodorant composition according to paragraphs A-P, wherein the composition further comprises an additional antimicrobial chosen from piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, or mixtures thereof.
R. The stick deodorant composition according to paragraph Q, wherein the additional antimicrobial comprises octenidine HCl.
S. The stick deodorant composition according to paragraphs A-R, wherein the composition comprises a melt point ≥55° C., preferably ≥57° C., more preferably ≥60° C., even more preferably ≥65° C. as determined by the Melt Point Test Method described herein.
T. The stick deodorant composition according to paragraphs A-S, wherein the composition comprises a % T at 600 nm of greater than 0, preferably ≥0.2, more preferably ≥0.4, more preferably ≥1, more preferably ≥ to 5, more preferably greater than ≥10, more preferably ≥12, more preferably ≥15, as determined by the Transmittance (% T) Test Method described herein.
U. The stick deodorant composition according to paragraphs A-T, wherein less than 50 micromoles acid per gram solvent titratable base are used to achieve the desired pH, preferably less than 45, and more preferably less than 40, as determined by to the Deodorant Titratable Base Method described herein.
V. The stick deodorant composition according to paragraphs A-U, wherein the composition further comprises from about from 0.1% to 10% fragrance, preferably from 1% to 5%.
W. Use of a stick deodorant composition comprising a solvent compartment comprising a solvent comprising at least one polyhydric alcohol and a blend of fatty acids comprising C16, C18, C20, and C22 carbon chain lengths; wherein the fatty acid blend comprises ≤10% C16 fatty acids, preferably ≤4%, more preferably ≤2%, to prevent or mitigate underarm odor, wetness, sweat, and/or microbial growth.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stick deodorant composition comprising:
a. a solvent compartment comprising a solvent comprising at least one polyhydric alcohol;
b. a blend of fatty acids comprising C16, C18, C20, and C22 carbon chain lengths;
wherein the fatty acid blend comprises less than about 30% C16 fatty acid, as a percentage of the total fatty acids;
wherein the deodorant composition is substantially free of aluminum.

2. The stick deodorant composition according to claim 1, wherein a weight ratio of the C18 to the C20+C22 is from about 1:1 to about 6:1.

3. The stick deodorant composition according to claim 1, wherein a weight ratio of the C16 to the C18+C22 fatty acids is less than about 1:3.

4. The stick deodorant composition according to claim 1, wherein the fatty acid comprises ≥30% of the C18 fatty acid, as a percentage of the total fatty acid.

5. The stick deodorant composition according to claim 1, wherein the fatty acid comprises ≥20% of the C20 fatty acid, as a percentage of the total fatty acid.

6. The stick deodorant composition according to claim 1, wherein the fatty acid comprises ≥10% of the C22 fatty acid, as a percentage of the total fatty acid salts.

7. The stick deodorant composition according to claim 1, wherein the solvent compartment comprises less than about 0.5% of the C16 fatty acid, by weight of the solvent compartment.

8. The stick deodorant composition according claim 1, wherein the composition comprises a pH from about 9 to 10.5.

9. The stick deodorant composition according to claim 1, wherein the composition comprises from about 50% to about 90% of the solvent.

10. The stick deodorant composition according to claim 1, wherein the solvent further comprises from about 1% to about 30% water.

11. The stick deodorant composition according to claim 10, wherein the weight ratio of water to polyhydric alcohol is from about 1:10 to about 1:1.

12. The stick deodorant composition according to claim 11, wherein the polyhydric alcohol and the water are miscible.

13. The stick deodorant composition according to claim 1, wherein the polyhydric alcohol is chosen from propylene glycol, dipropylene glycol, tripropylene glycol, low molecular weight polypropylene glycols, 1,3 propanediol, ethylene glycol, diethylene glycol, triethylene glycol, butylene glycol, hexylene glycol, trimethylene glycol, 1,2 pentanediol, 1,2 hexanediol, butantediol, glycerol, sorbitol, and polyethylene glycol including PEG-4 and PEG-8, or mixtures thereof.

14. The stick deodorant composition according to claim 1, wherein the composition further comprises an additional antimicrobial chosen from piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, or mixtures thereof.

15. The stick deodorant composition according to claim 14, wherein the additional antimicrobial comprises octenidine HCl.

16. The stick deodorant composition according to claim 1, wherein the composition comprises a melt point ≥ about 55° C.

17. The stick deodorant composition according to claim 1, wherein the composition comprises a % T at 600 nm of greater than 0.

18. The stick deodorant composition according to claim 1, wherein less than 50 micromoles acid per gram solvent titratable base are used to achieve the desired pH.
